(19) United States
(12) Patent Application Publication
(10) Pub. No.: US 2002/0016007 A1
(43) Pub. Date: Feb. 7, 2002

(54) Micro-analysis

(76) Inventors: Sean Criaplan Keeping, Shortlands (GB); Dieter Binz, Ladenburg (DE); Albrecht Vogel, Stutensee (DE)

Correspondence Address: LARSON & TAYLOR, PLC, 1199 NORTH FAIRFAX STREET, SUITE 900, ALEXANDRIA, VA, 22314, US

(21) Appl. No.: 09/867,740

(22) Filed: May 31, 2001

(30) Foreign Application Priority Data
May 31, 2000 (GB) 0013213.4

(51) Int. Cl. G01N 33/558

(52) U.S. Cl. 436/514

(57) ABSTRACT

In a micro-channel analysis device, the problem of air bubble formation is alleviated by provision of a debubbling zone in which the fluid to be analyzed is heated to a temperature greater than the micro channel and dissolved gases are vented.

The present invention relates to analysis of a fluid, particularly micro-fluidic analysis in which fluid is caused to flow along micro-scale channels for example formed in a wafer or other substrate. An example of such a micro-scale analysis device may be found in our earlier application EP-A-844478, the entire disclosure of which is incorporated herein by reference.

Micro-fluidic analysis is potentially attractive as it promises to reduce the amounts of reagents required for analysis and hence reduce costs of analysis. However, design of successful micro-fluidic handling devices may require solution of problems which are not present in larger devices.

A problem we have found with such devices is that air dissolved in the fluid to be analysed may form bubbles causing obstructions to the fluid flow and impeding reliability of operation of the device. In an attempt to reduce or solve this problem, we have found that careful shaping of the channels, in particular minimising or smoothing sudden changes in direction or channel size can reduce the tendency for the channels to be blocked by air bubbles, However, problems can still occur and implementing such smoothing may complicate the manufacture or restrict the ability to perform analysis in a desired manner.

The present invention proposes an alternative solution which may remarkably reduce the problem of bubble formation in a micro-fluidic analysis device.

According to a first aspect, the present invention provides a method of operating a micro-channel fluid analysis device comprising receiving fluid to be analysed and passing the fluid to be analysed through the micro channel analysis device having at least one micro channel through which the

fluid is arranged to flow characterised in that the fluid to be analysed is passed through a debubbling zone maintained at a temperature substantially higher than the temperature of said micro channel so that dissolved gases in the fluid are discharged in the debubbling zone in preference to said micro channel.

Thus, by controlling temperature in this way, a remarkable reduction in bubble formation can be effected, which may reduce or eliminate the need for certain shaping and sizing precautions in the micro channel structure.

Preferably, the debubbling zone is vented to allow discharged gases to escape.

Preferably, the fluid is passed upwardly, preferably through an inclined channel, to a relatively warm portion of the debubbling zone to a point at which dissolved gases can escape and then downwardly towards the micro channel analysis device.

The method may include heating the debubbling zone and/or cooling the micro channel analysis device. Whilst the debubbling zone and analysis device may be positively heated or cooled, in some embodiments, the debubbling zone may simply be provided at a portion which will be hotter than the channel. The micro channel analysis device preferably comprises a substrate having a plurality of channels formed therein.

According to a second aspect, the invention provides apparatus for analysing a fluid comprising means for receiving a fluid to be analysed and a micro channel analysis device including at least one micro channel through which the fluid is arranged to flow, characterised by debubbling means located in the fluid path between the receiving means and the micro channel analysis device and arranged to be maintained at a temperature substantially higher than the micro channel.

The apparatus preferably includes means for heating the debubbling means. The debubbling means preferably includes an upwardly inclined conduit for directing the received fluid to a zone at which dissolved air can escape from the surface of the fluid and a downwardly inclined or substantially vertical conduit for directing fluid from said zone towards the micro channel analysis device.

An embodiment of the invention will now be described, by way of example only, with reference to the accompanying diagram.

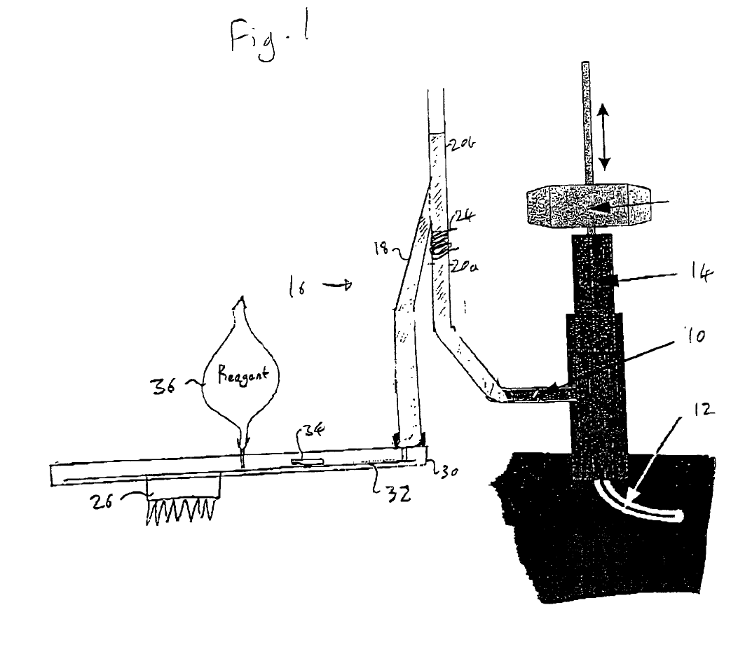

FIG. 1 depicts schematically an analysis device according to an embodiment.

Referring to FIG. 1, an analysis device comprises a fluid inlet 10 for receiving a fluid to be analysed which, in this embodiment, is drawn through a filter assembly 12 such as that described in our co-pending application which bears the attorney reference IK/20664, the disclosure of which is incorporated herein by reference. The fluid is pumped by pump 14 to debubbler 16 which comprises a substantially vertical conduit 20a, 20b having a junction with a downwardly inclined conduit 18, prefarably separated from the downwardly extending conduit by a mesh screen of size small enough to inhibit bubbles passing. The upper portion of the substantially vertical conduit 20a is open to the atmosphere and the lower portion 20b receives fluid which may contain bubbles, the conduit 18 leading to micro channel analysis device 30. The analysis device may be as described, for example, in our application EP-A-844478 and typically comprises a substrate 32, typically a silicon wafer, having a plurality of channels defined therein and a plurality of pumps 34 and other analysis means thereon.

By micro channel analysis device in this specification (which term includes the claims), we mean to include devices having small fluid flow channels which would be liable to blockage by bubble formation. Typically the channels will have a maximum dimension of less than 2 mm and ideally less than 1 mm. The invention is advantageous in allowing smaller channels to be used, and hence a reduction in the amount of fluid required for analysis without risk of blockage due to air bubble formation. Preferred micro channel analysis devices will have dimensions of less than 0.5 mm, for example between about 1 and 500 microns or of the order of 10-100 microns, formed, for example, by etching of a substrate such as a silicon or ceramic wafer, or in some cases in a plastic or glass substrate.

A supply of reagents 36 for use in analysis may be provided, and the arrangement of reagents may be based on the apportionment of reagents described in our concurrently filed application which bears the attorney reference IK/20666, the disclosure of which is incorporated herein by reference.

Particularly in the region of the junction of the conduit, a heater 24 is provided to heat fluid to a temperature within a predetermined range. It is a preferred feature that the heater is provided in the vicinity of a junction between an upwardly directed inlet conduit and a downwardly directed outlet conduit. The device also has temperature regulating means 26, which may include a Peltier heat pump for heating and/or cooling arranged to maintain the micro channel analysis device substantially within a predetermined temperature range. In some cases, there may “naturally” be a hottest point of the analysis device, for example in the vicinity of control or power electronics and the debubbler may be placed in this location so that positive heating is not required; indeed, the debubbling zone may serve a useful additional function of stabilising the temperature or cooling a portion of the device, although the fluid flow volumes will not normally be sufficient to provide reliable cooling by this means alone.

The heater 24 may be a simple heating coil, More preferably, a device whose temperature may be readily controlled, for example a semiconductor, such as a transistor is used to generate heat and the current through the heating element is controlled by means of a feedback circuit to maintain a substantially constant temperature. The heater may be placed to deliver heat primarily to the inlet conduit 20a, preferably just below the junction with the outlet conduit 18, so that any bubbles forming in the heating zone rise vertically direct to vent conduit 20b. As a further alternative, the heating zone may deliver heat primarily to a portion of the inlet conduit so that the inlet fluid is hottest and bubbles have more time to form in the inlet conduit and be vented before reaching the outlet conduit (this is particularly advantageous in conjunction with a capillary trap at the top of the outlet conduit). Where certain elements are provided on a replaceable cartridge, for example as described in our concurrently filed application which bears the attorney reference IK/20665, the disclosure of which is incorporated herein by reference, it is preferred for the heating element to be located on the base unit.

Certain portions of the micro channel analysis device may be heated or cooled to higher or lower localised temperatures than the majority of the device and it is possible that certain points will be as hot as, or even hotter than, the temperature of the debubbling zone. However, it is greatly preferred that the hottest point in the fluid path is in the debubbling zone. Typically, this will entail maintaining the debubbling zone at least 5° C. and preferably at least 10° C., more preferably at least about 20° C. or even 30° C. hotter than the micro channel analysis device. The higher the temperature, the more effective will be the debubbling. However, with higher temperatures, greater amounts of energy will be consumed and there is a risk of denaturing or modifying components within the fluid to be analysed (this risk may be acceptable in many applications, for example in certain analyses of waste water).

Typically and preferably, the debubbling zone is maintained above about 40° C., typically in the range of 40° C. to 95° C., or about 70° and the micro channel analysis device is maintained below 40° C. typically at most 37° C., and preferably about 20° C. Preferably, the temperature of the fluid decreases substantially monotonically from the debubbling zone as it flows through the micro channel analysis device. Preferably areas of the device which present exceptions to the general decrease in temperature from the debubbling zone have provision for venting of gases.

The debubbling zone may heat the liquid to a temperature sufficient to sterilize it, for example by heating to at least about 70° C. In preferred embodiments, however, the liquid is pre-filtered and should be sterile prior to passing to the debubbling zone, so this may not be necessary, or may provide an added precaution against microbial contamination.

A capillary trap may be provided between the debubbling zone and the micro channel to minimise the risk of entrained bubbles being carried into the micro channel. This may advantageously be provided on the upper portion of a downwardly extending conduit from the debubbling zone, for example at the upper portion of conduit 20b, or preferably below the heater if the heater extends down the conduit 20b. Advantageously, the capillary trap may take the form of a fine mesh screen, preferably of pore size smaller than the micro-channel dimensions (for example as indicated above), of a size effective to inhibit passage of bubbles formed in the debubbling zone.

The apparatus may include means for selecting a service or debubbling mode in which the analysis device (the micro channel) is cooled to a temperature below normal operating temperature to re-dissolve any bubbles which have formed. This feature may be independently provided or provided as an independent method aspect in a method of controlling the temperature of a micro channel analysis device arranged for operation at a first temperature comprising cooling the device to a second temperature, lower than the first, to re-dissolve a gas bubble, preferably in response to a determination that a blockage has occurred due to bubble formation, for example based on a measure of fluid flow, or periodically.

Modifications will be apparent and each feature disclosed herein, particularly preferred features, may be independently provided, unless otherwise stated. The appended abstract is incorporated herein by reference.

1. A method of operating a micro-channel fluid analysis device comprising receiving fluid to be analysed and passing the fluid to be analysed through the micro channel analysis device having at least one micro channel through which the fluid is arranged to flow characterised in that the fluid to be analysed is passed through a debubbling zone maintained at a temperature substantially higher than the temperature of said micro channel so that dissolved gases in the fluid are discharged in the debubbling zone in preference to said micro channel.

2. A method according to claim 1 wherein the debubbling zone is vented to allow discharged gases to escape.

3. A method according to claim 1 wherein the fluid is passed upwardly, preferably through an inclined channel, to a relatively warm portion of the debubbling zone to a point at which dissolved gases can scape and then downwardly towards the micro channel analysis device.

4. A method according to claim 1 including heating the debubbling zone.

5. A method according to claim 1 including cooling the micro channel analysis device.

6. A method according to claim 1 wherein the debubbling zone is maintained at least 5 degrees Celsius hotter than the micro channel analysis device.

7. A method according to claim 1, wherein the debubbling zone is maintained at a temperature sufficient substantially to sterilize the fluid.

8. A method according to claim 1, wherein the fluid is passed through a capillary trap or screen to inhibit passage of bubbles between the debubbling zone and the micro channel.

9. Apparatus for analysing a fluid comprising means for receiving a fluid to be analysed and a micro channel analysis device including at least one micro channel through which the fluid is arranged to flow, characterised by debubbling means located in the fluid path between the receiving means and the micro channel analysis device and arranged to be maintained at a temperature substantially higher than the micro channel.

10. Apparatus according to claim 9 including means for heating the debubbling means.

11. Apparatus according to claim 9 wherein the debubbling means includes an upwardly directed, peferably inclined, conduit for directing the received fluid to a zone at which dissolved air can escape from the surface of the fluid and a downwardly directed, preferably substantially vertical, conduit for directing fluid from said zone towards the micro channel analysis device.

12. Apparatus according to claim 9, including a capillary trap or screen for inhibiting passage of bubbles.

13. Apparatus according to claim 9, including a temperature controller for controlling the temperature of the debubbling zone to be higher than that of the micro channel.

14. A micro fluidic analysis device substantially as herein described or as illustrated in the accompanying drawing.
